(19) United States
(12) Patent Application Publication
(10) Pub. No.: US 2017/0325975 A1
(43) Pub. Date: Nov. 16, 2017

(54) COOLING SYSTEM AND METHOD FOR A PROSTHETIC SOCKET

(71) Applicant: Vivonics, Inc., Bedford, MA (US)

(72) Inventors: Kristen LeRoy, Waltham, MA (US); Ian Cohen, Allston, MA (US); Gordon B. Hirschman, Cohoes, NY (US); Thane R. Hunt, Colchester, CT (US); Kevin E. Keough, Sharon, MA (US); Carlos Martinez Luna, Boylston, MA (US); Todd R. Farrell, Waltham, MA (US); Jennifer L. Johansson, Wayland, MA (US); Brendan LaBrecque, Middleton, MA (US)

(73) Assignees: Vivonics, Inc.; Liberating Technologies, Inc., Holliston, MA (US)

(21) Appl. No.: 15/590,679

(22) Filed: May 9, 2017

Related U.S. Application Data
(60) Provisional application No. 62/334,758, filed on May 11, 2016.

(51) Int. Cl. A61F 2/80; A61F 2/78; A61F 7/00; A61F 5/01

(52) U.S. Cl. CPC A61F 2/80; A61F 2/7812; A61F 2007/0051; A61F 5/01

(57) ABSTRACT

A prosthetic socket cooling system and method includes a strap for coupling about a limb fitted with a prosthetic socket. One or more thermally conductive heat spreaders are associated with the strap and may be at least partially seated underneath the prosthetic socket. A heat extraction subsystem is coupled to at least one of the lengthy heat spreaders and carried by the strap. In one version, heat spreaders may form sections of the strap.

### RELATED APPLICATIONS

This application claims benefit of and priority to U.S. Provisional Application Ser. No. 62/334,758 filed May 11, 2016, under 35 U.S.C. §§119, 120, 363, 365, and 37 C.F.R. §1.55 and §1.78, which is incorporated herein by this reference.

### GOVERNMENT RIGHTS

This invention was made with government support under W81XWH-13-1-0453 awarded by the U.S. Army. The government has certain rights in the invention.

### FIELD OF THE INVENTION

This invention relates to a cooling system and method for a prosthetic socket.

### BACKGROUND OF THE INVENTION

Nearly 2 million people are living with limb loss in the United States. A significant portion of both civilians and soldiers who undergo amputation are now being fitted with state of the art prosthetic devices. Improvements in prosthetic limb function have outpaced improvements to the comfort of the devices. Prosthetic sockets typically include a hard outer shell that functions as a mechanical interface between the residual limb and prosthetic limb, e.g., a foot, a hand, and the like. A silicone liner up to about 1 cm thick may be worn over the residual limb for cushioning and to improve connection to the prosthesis. Layers of socks may also be worn over the liner to maintain socket fit as the limb experiences natural changes in residual limb volume. Heat and moisture trapped by these non-breathable and thermally insulating materials may create a warm, moist, and adverse environment.

The trapped heat and perspiration may lead to potential skin problems of the residual limb such as folliculitis, friction blisters, bacterial growth, and the like. In one survey of transfemoral amputees, heat and perspiration inside the socket was reported by 72 % of the survey participants as the most common cause for a reduced quality of life. Similarly, poorly managed moisture at the interface between the residual limb and the inner prosthetic socket and/or liner may lead to skin irritation and infections which may decrease the usability of the prosthesis. Elevated temperatures in the prosthetic socket may also lead to increased sweating and friction, skin damage, discomfort, and reduced quality of life.

Studies have found increases in socket temperature for a period as short as 30 minutes of walking after the prosthesis was donned. It was also found that temperatures remained elevated long after activity cessation. Even a rest period greatly exceeding the duration of the preceding activity period may be insufficient to return the limb to its initial temperature. Studies also suggest that a modest temperature increase of only 2° C. may be responsible for reports of thermal discomfort by amputees. Therefore, a small amount of activity may cause the socket temperature to elevate and remain at an uncomfortable level for an extended period of time which may lead to decreased wear times.

In summary, an uncomfortable or non-performing socket/residual limb interface due to temperature increase in the socket may decrease prosthesis use among amputees who want to remain active in their civilian and military lives.

Several prior publications propose prosthetic cooling systems integrated with the prosthetic socket. See, for example, U.S. Patent Publication 2016/0030207; U.S. Pat. No. 9,358,138; U.S. 2015/0105865; US 2016/0030207; U.S. Pat. No. 6,123,716; and WO 2017/004540 all incorporated herein by this reference.

### SUMMARY OF THE INVENTION

Featured is a prosthetic socket cooling system (or thermal management device) comprising a strap for coupling about a limb fitted with a prosthetic socket. One or more lengthy thermally conductive heat spreaders may extend from the strap and, in one example are at least partially seated underneath the prosthetic socket. A heat extraction subsystem is coupled to at least one of the lengthy heat spreaders.

The heat spreaders may be rectangular in shape and may be made of copper, aluminum, or graphite or other thermally conductive material. In one version, the heat spreaders include one or more heat pipes. In some examples, the strap includes a thermally conductive material.

The heat extraction subsystem may include a finned heat sink and a fan positioned to urge air through the fins of the heat sink. The subsystem may further include a TEC coupled between the heat spreader and the heat sink. In some examples, the heat extraction subsystem further includes a user interface, an electronic section, and a battery for powering the TEC, the fan, and the electronics section. A heat extraction subsystem device may further include a housing about the fan, the TEC, the heat sink, the user interface, the electronics section, and the battery. The electronics section may further include a controller subsystem and one or more temperature sensors. The controller may be configured to operate the fan and/or the TEC based on signals from the user interface and the temperature sensors.

Also featured is a method of cooling a prosthetic socket. A plurality of spaced lengthy thermally conductive heat spreaders are strapped about a limb fitted with a prosthetic socket. The lengthy thermally conductive heat spreaders are placed underneath the prosthetic socket. A heat extraction subsystem device is coupled to at least one of the lengthy heat spreaders. The device is operated to drive heat from the prosthetic socket to the external environment via the heat extraction subsystem.

Also featured is a system for cooling a prosthetic socket on a limb. A strap is coupled about the limb above the prosthetic socket. One or more thermally conductive heat spreaders are associated with the strap. A heat extraction subsystem is carried by the strap. In one version, the heat spreaders extend from the strap and can be disposed underneath the prosthetic socket. In another version, the heat spreaders are a part of the strap and/or form sections of the strap.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic three-dimensional view showing one example of a prosthetic socket cooling system;

FIG. 2 is a schematic view showing the heat spreaders of the prosthetic socket cooling system of FIG. 1 in place underneath a prosthetic socket;

FIG. 3 is a schematic view showing another example of a prosthetic socket cooling system;

FIG. 4 is a schematic view showing a curved limb conforming heat spreader in accordance with aspects of the invention;

FIG. 5 is a schematic view showing the primary components associated with an example of a heat extraction subsystem for the prosthetic socket cooling system;

FIG. 6 is a schematic view an assembled heat extraction subsystem;

FIG. 7 is another schematic view of a heat extraction subsystem;

FIG. 8 is schematic cross sectional view showing the air channels formed by the housing sections of heat extraction subsystem;

FIG. 9 is a schematic view showing an example of a heat extraction subsystem fan mounted adjacent a heat sink and TEC;

FIG. 10 is a schematic view showing an example of the invention where a heat spreader includes heat pipes;

FIG. 11 is a block diagram showing the primary components associated with a prosthetic socket cooling system;

FIG. 12 is a another block diagram showing additional details associated with an example of a prosthetic socket cooling system;

FIG. 13 is a flow chart showing the main algorithm executed by the controller of FIGS. 11 and 12;

FIG. 14 is a schematic view showing one example of a cooling control loop algorithm executed by the controller of FIGS. 11 and 12; and

FIG. 15 is a flow chart showing another example of a cooling control loop executed by the controller of FIGS. 11 and 12;

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

A prosthetic socket cooling system in one or more embodiments of this invention is located alongside the prosthetic socket and allows the user to control the temperature within the socket and the residual limb to effectively reduce or eliminate the problems associated with the elevated temperature in the prosthetic socket discussed in the Background section above. The system preferably includes one or more heat spreaders and a heat extraction subsystem. The heat spreader is preferably made of a sheet of high thermally conductive material, e.g., copper, aluminum, graphite, stainless steel, or similar type of metal material which meets the heat transfer requirements of a given patient that draws or absorbs heat from a large area of the residual limb on the prosthetic side and transports or dissipates the heat energy. The heat extraction subsystem draws or absorbs heat from the heat spreader and discharges or dissipates the heat to the environment side external to the socket.

The heat spreader preferably transfers heat from a relatively large area of the residual limb to the heat extraction subsystem through a relatively small cross-sectional area. The heat spreader may range in length depending on the diameter and length of the residual limb, e.g., the range of about 4″ to about 10″, although the heat spreader may be longer or shorter as needed. Typically, the heat spreader is between 1.5 and 4 inches wide and between 0.02 and 0.05 inches thick.

The heat extraction subsystem may vary in size depending on the particular needs of the patient, e.g., about 2″ in length and width, although the subsystem may be larger or smaller as needed. One or more heat extraction subsystem devices may be used for a single heat spreader and a plurality of heat spreaders may be associated with a single heat extraction subsystem device. The one or more heat spreaders may be shaped as an elongated rectangle, oval, square, circle, or other shape based on the individual needs of the patient (disclosed below). One or more heat spreaders may be oriented such that they wrap around the limb circumferentially and/or run axially down the length of the limb (also disclosed below).

One or more heat spreaders may be attached to one or more components of a heat extraction subsystem using a thermal adhesive, a mechanical attachment, a combination thereof, or similar type of attachment technique. The mechanical attachment may include press-fitting the heat spreader into corresponding grooves in a heat extractor component, clamping between a pair of plates or between one plate and the body of a heat extractor component, or attaching it directly to the heat spreader using thermal tape or thermal pads, welding (e.g., by friction, deposition, resistance spot welding, and the like), brazing, direct attachment using pins, screws, or related hardware, snap fitting, or other known methods of mechanical attachment known to those skilled in the art.

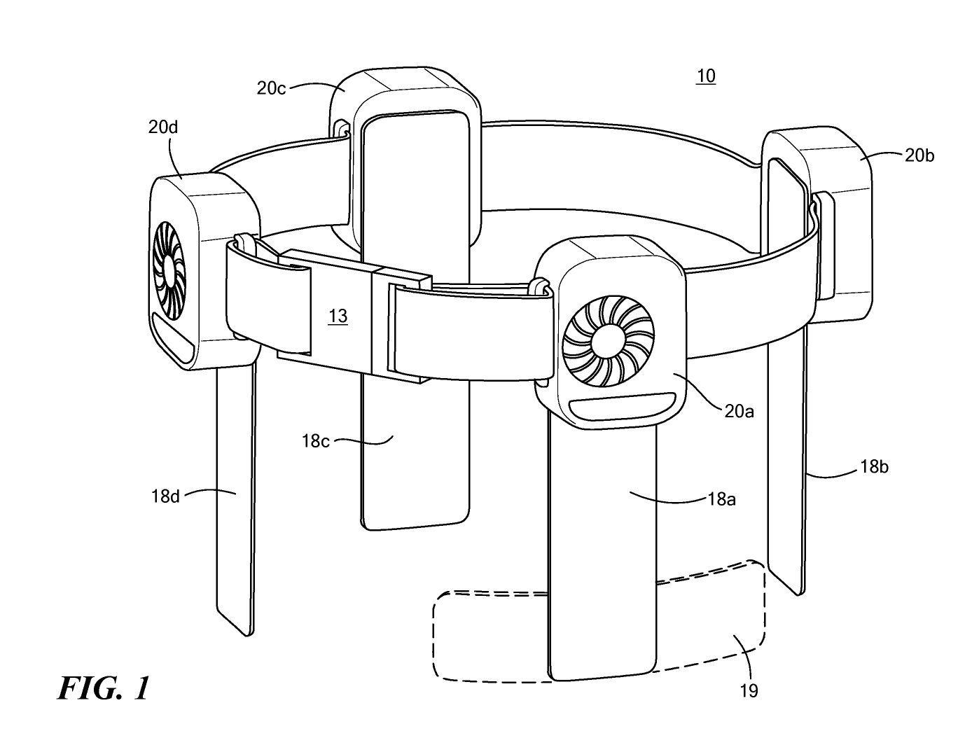

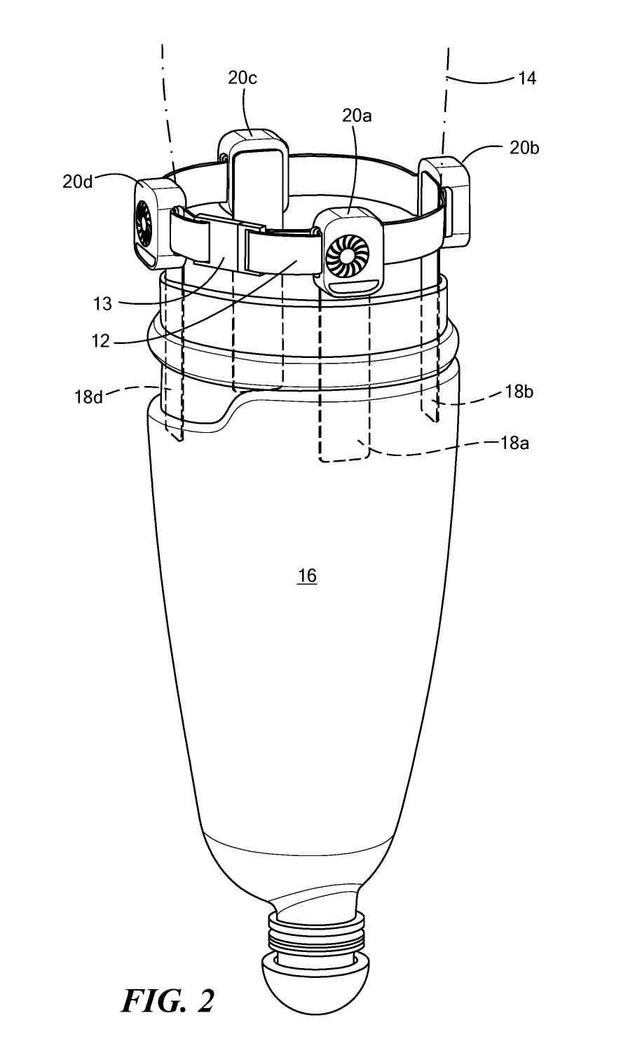

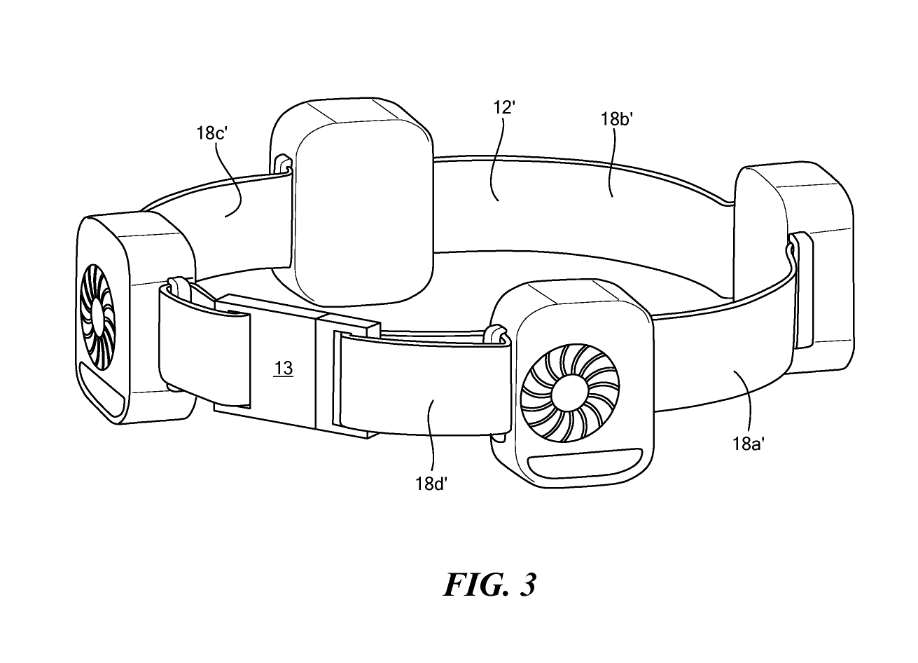

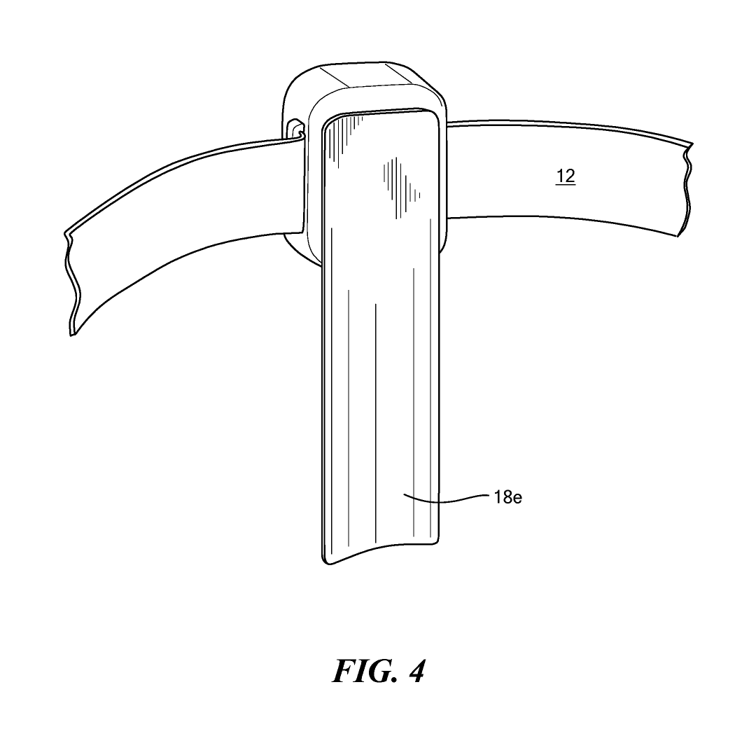

FIG. 1 shows an example of a prosthetic socket cooling system 10 including a strap 12 with buckle 13 for coupling about a limb 14, FIG. 2 fitted with prosthetic socket 16. Other methods of attaching the strap to the limb include hook and loop fasteners, snaps, hooks, or other means of mechanical attachment known to those skilled in the art. Extending from strap 12 (but not necessarily directly coupled thereto) are a plurality of spaced, lengthy, thin thermally conductive heat spreaders 18a, 18b, 18c, and 18d. A smaller number of larger, or a larger number of smaller width heat spreaders may be used. In one example, only one heat spreader located on the posterior of the leg is used. The one or more heat spreaders seat underneath prosthetic socket 16 (e.g., under a sock worn by the user, between the sock and the prosthetic socket liner, between the liner and the socket, between the liner and the limb, or the like). Rounded corners are preferred. In some examples, the heat spreaders are rectangular or elliptical in shape. In the design shown in phantom for heat spreader 18a, FIG. 1, the heat spreader may be T-shaped with concave head 19 conforming to the limb. The strap 12 may also be made of or include thermally conductive material for cooling. Indeed, in the design of FIG. 3, the one or more heat spreaders 18a′ 18b′, 18c′ and 18d′ are sections of the strap. This strap may be worn with or without a prosthetic. The cooling band may be worn near the groin where the femoral artery is closest to the surface of the skin. The band may also be worn or on the arm. In the design of FIG. 4, a single large posteriorly located curved concave heat spreader 18e is used conforming to the limb.

There may be one or more heat extraction subsystems devices 20a, 20b, 20c, 20d, for each heat spreader although not every heat spreader may require its own device. The devices are typically disposed at the top of each heat spreader 18.

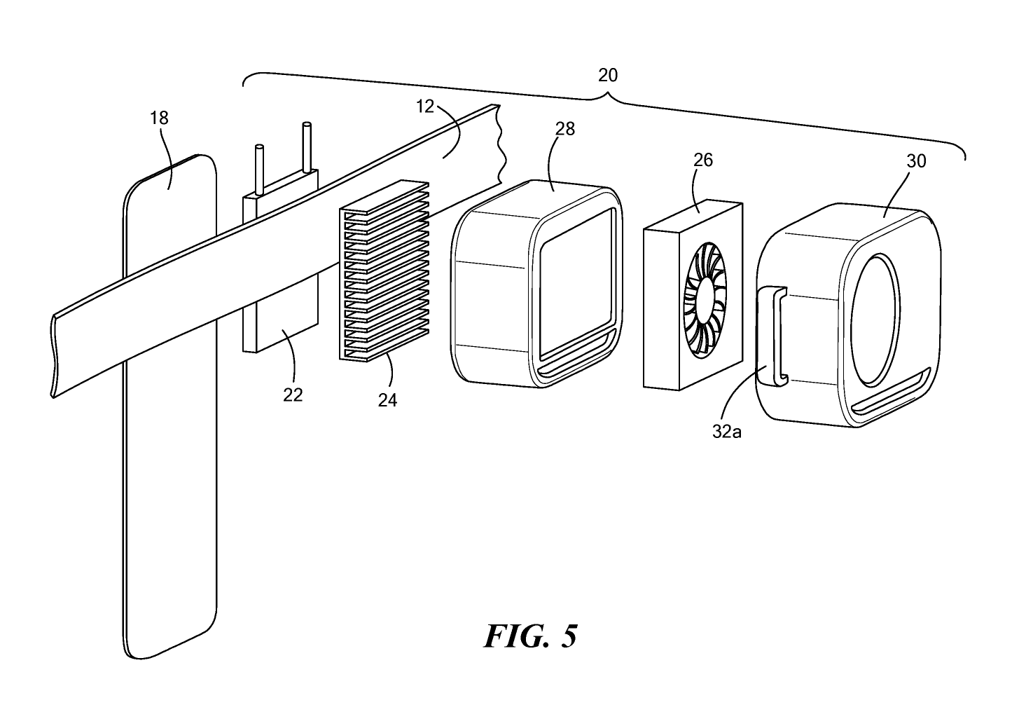

In FIG. 5, a heat extraction subsystem includes an optional Peltier-effect thermal electric cooler (TEC) 22 disposed between heat spreader 18 and finned heat sink 24 and fan 26. The fan 26 is configured to move air between the fins of heat sink 24 out to the environment or to blow air over the fins of heat sink 24. In other embodiments, the heat extraction subsystem device includes a TEC and a fan without a heat sink.

Strap 12 may be disposed between TEC 22 and heat sink 24 and made of a flexible, thermally-conductive material or provided with a cutout or thermally conductive area to allow effective heat transfer between TEC 22 and heat sink 24. One heat extraction subsystem device 20 includes housing sections 28 and 30 to form a housing for the device. Housing section 30 may include opposing slots 32a, 32b for strap 12. In some embodiments, TEC 22 is not used.

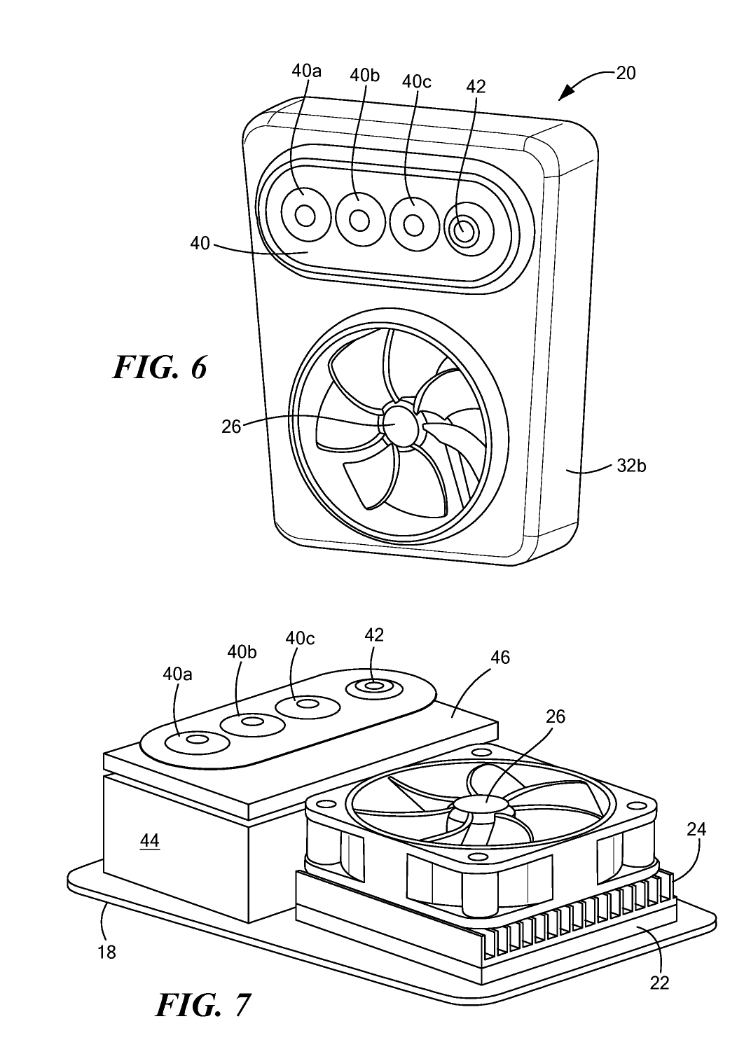

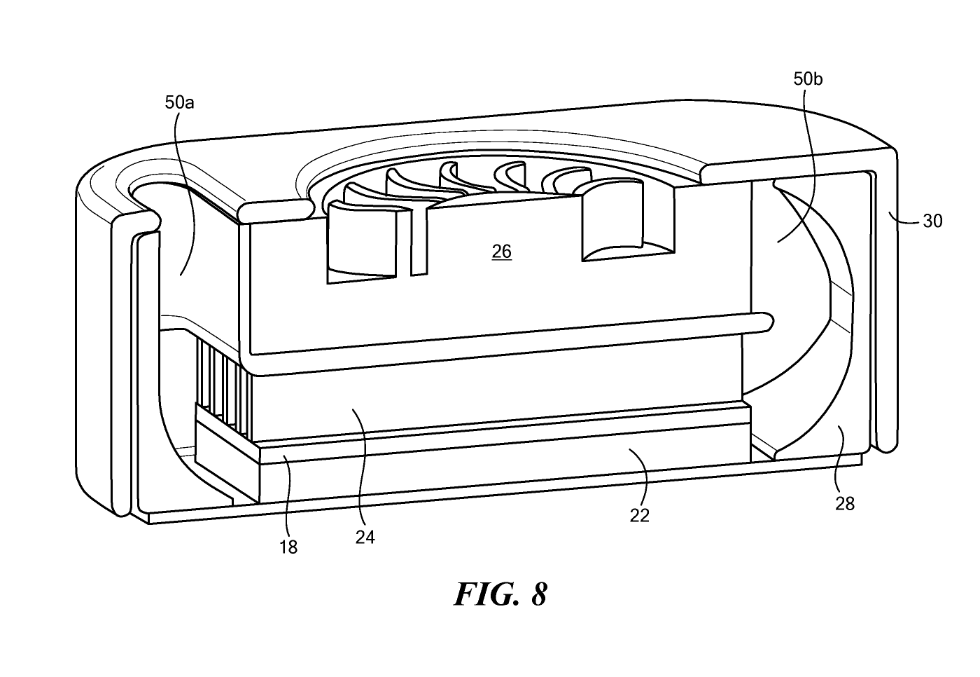

In some embodiments, the heat extraction subsystem device includes user interface 40 with temperature control buttons 40a and 40b and on/off switch 40c, FIG. 6. Charging port 42 may be included for charging battery 44, FIG. 7. An electronics section 46 (e.g., a populated printed circuit board) may further be included. See also FIG. 8 where housing sections 28, 30 form air flow channels 50a and 50b.

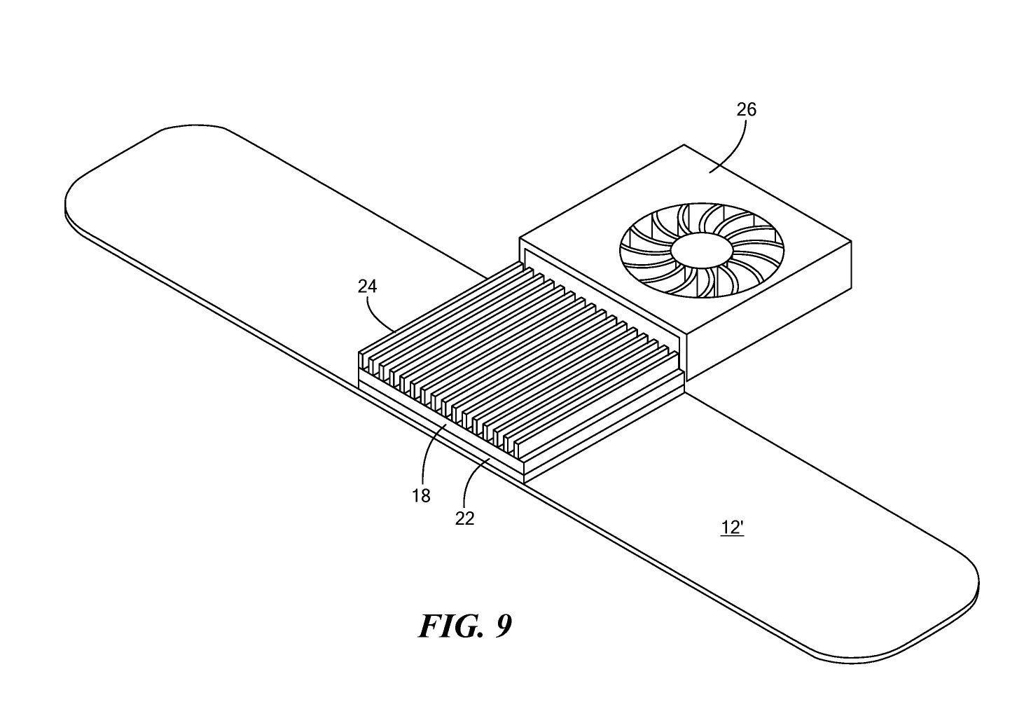

In FIG. 9, heat sink 24 is mounted to the heat spreader 12′ and a centrifugal/blower fan 26 is located to the side of heat sink 24 and used to blow air over the fins thereof. This side-by-side configuration of the fan and heat sink could also be used in other configurations of the device such as that shown in FIG. 1. Heat spreader 12′ may be a portion of the strap or connected to the strap.

In the design of FIG. 10, the heat spreader 18′ includes heat pipes. Heat pipes are tubes made of a thermally conductive material, e.g., copper or a similar high thermally conductive material that are sealed on the ends and filled with a wicking material and a working fluid under partial vacuum such that wicking material exists at both liquid and gaseous state over the operating temperature range. The working fluid expands to the gas state at the hot side of the heat pipe (absorbing the latent heat of vaporization from the external hot area) then condenses to a liquid at the cold side releasing the latent heat. The heat pipes may have a circular cross section or may be flattened as shown to increase contact area at the limb interface. One or more heat pipes may be used to form the heat spreading surface, or to interface one or more existing thermal surfaces over a distance. The heat spreader configured as heat pipes may include a clamping mechanism 43 as shown with a platform that preferably includes threaded holes, snaps, ridges, and the like, for coupling it to the heat extractor.

In one example the heat spreader configured as heat pipes may be coupled to the heat spreader with an attachment mechanism shown, e.g., screws, an adhesive, a snap fit, a press fit, or similar techniques as discussed above. Thermal contact may be improved between the heat extractor and heat spreader with a conductive epoxy, conductive tape, or the like.

In one design, the one or more heat spreaders configured as heat pipes may include a heat extractor attachment with a platform that preferably includes threaded holes, snaps, ridges, and the like, for coupling the heat pipes to the heat extractor. Thermal contact may be improved between the sink and spreader with a conductive epoxy, conductive tape, or other known methods of thermal interfacing known to those skilled in the art.

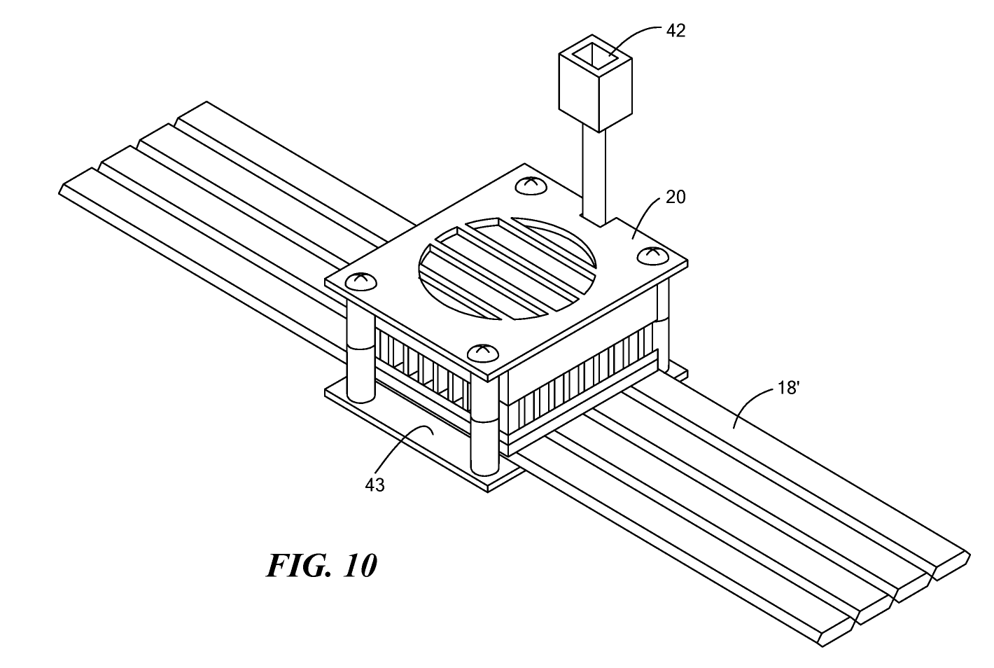

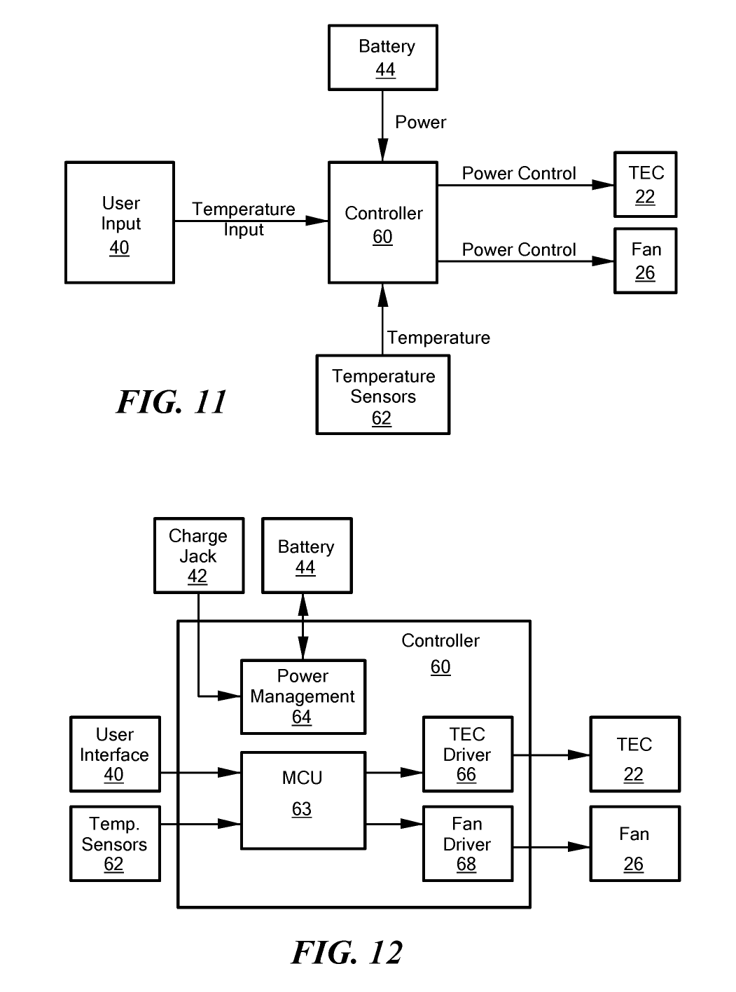

As shown in FIG. 11, the electronic section may include controller subsystem 60 (e.g., one or more microprocessors, microcontrollers, field programmable gate arrays, or other logic devices or the like) may be configured (e.g., programmed) to operate fan 26 and/or the TEC 22 if present) based on the outputs of user input 40 and, optionally, one or more temperature sensors 62.

Power may be applied to the TEC via the power adapter and the power source. The power source applies a voltage across two dissimilar metals within the TEC to create a temperature difference via the Peltier effect which increases the rate of heat transfer from the heat spreader to the heat sink. The TEC transfers more heat to the fins which further increases the rate of heat exchange between the fins and the environment side. The TEC functions to reduce the temperature inside the socket. The power source coupled to the power adapter may be an external component linked with a wire or packaged together in the same housing. The power source may control the power sent to the TEC. A thermostat may be used to automatically adjust the power to the TEC to achieve an actively regulated temperature. The power source may be adjusted to control the power sent to the TEC and fan. The TEC and fan may be independently regulated with distinct current and voltages. The power source may be coupled to controls that allows the user to adjust the temperature set point of the thermal management device. The user interface 4.0 preferably includes a lower temperature button, an increase temperature button, an on/off button, and a charging connection as shown. The user interface allows the user to plug in the thermal management device to charge up a rechargeable battery (not shown), turn the thermal management device on or off, increase or lower the temperature to set temperature thresholds (discussed below), and provide control of the other various functions of the thermal management device. To increase the set point temperature, the user may press the increase temperature button. To decrease the set point temperature, the user may press the lower temperature button. In order to turn the device on an off, the user may press and hold the on/off button for three seconds for the power state change to occur. A battery, integrated with a heat extraction subsystem device or located externally, provides power to the electronic components.

In one design, a printed circuit board (PCB) includes all the necessary electrical components known to those skilled in the art to manage the power of the heat extraction subsystem device, compute, and send/receive control signals to/from peripheral devices, e.g., the fan and the TEC shown. The PCB may include a controller, which includes a microprocessor unit (MCU), 63, FIG. 12 which may be programmed to manage the temperature within the prosthetic socket with input from temperature sensors as well as the temperature control input of the user interface. The PCB may also include a power management circuitry 64 which manages the input and charging of the battery as well as routing of power to the rest of the board. TEC and fan drivers, 66 and 68 control power to the TEC and fan.

Battery 44 may be a rechargeable lithium ion battery, or similar type battery. There are many different battery chemistries that may be suitable for the thermal management device for a prosthetic socket of one or more embodiments of this invention, e.g., lithium polymer, nickel-cadmium, and the like. The battery preferably powers all the various components. The battery may be charged via a charging connection on the device as discussed above or may be removable so it may be replaced with a fully charged battery. The charger for the device may be connected to an AC outlet and contains the necessary circuitry to correctly charge the device.

In some designs, temperature sensors 62, e.g., thermocouples, thermistors, or similar type device may be placed in preferred locations within prosthetic socket or the thermal management device to measure and evaluate the temperature of the residual limb of the user and the device to ensure safety and efficiency. The sensors may be placed to measure the temperature within the socket, the temperature of the cold side of the TEC, the temperature of the hot side of the TEC, and/or the ambient temperature of the environment outside of the heat extraction subsystem device. A thermistor typically includes a metallic oxide encapsulated within a bead of glass or similar material. When the temperature of the thermistor varies, the resistance of the internal material changes, which can be interpreted as a change in voltage when the appropriate peripheral circuitry and control methods are used. This voltage is then mathematically calibrated to a unit of temperature via a linear or nonlinear equation.

A thermocouple typically includes two lengths of dissimilar metals mechanically bonded at either end. When one material, e.g., the hot junction, becomes warmer than the other, e.g., cold junction, a voltage potential develops. This voltage may be amplified and adjusted as necessary through the use of the appropriate peripheral circuitry and control methods. This voltage is then mathematically calibrated to a unit of temperature via a linear or nonlinear equation.

In one example, current may be reversed to the TEC in order to provide heating for the prosthetic limb or temporarily slow the rate of cooling if the controller determines that cooling is occurring too rapidly.

The battery may provide power to the controller and peripheral components of the system discussed above. The user interface discussed above allows the user to raise or lower the desired temperature set point. The set point is then sent to the controller and is used to drive the control algorithms. The temperature sensors may capture the temperatures within the socket, on both sides of the TEC, as well as the ambient temperature, to determine the power needs of the TEC and fan.

The battery may provide power to the controller and peripheral components as discussed above. The user input allows the user to raise or lower the desired temperature set point. The set point is sent to the controller and is used to drive the control algorithms. The temperature sensors capture the temperatures within the socket and the ambient temperature to determine the power needs of the fan.

The battery may provide power to the controller and the peripheral components as discussed above. The user interface may include at least two buttons discussed above that allow the user to adjust the level of desired cooling. Temperature sensors are preferably placed at strategic locations in the prosthetic socket or in the thermal management device to monitor temperature for safety as well as efficiency of the cooling system. Both of these inputs are provided as feedback to the microprocessor (MCU) to determine optimal control signals for both the fan and TEC to accomplish the desired temperature. These control signals are then sent to the TEC and fan drivers to convert the control signals into the electrical power needed to drive the TEC and fan.

The battery may also provide power to the controller and the peripheral components, as discussed above. The user interface may include temperature control buttons as discussed above which allow the user to adjust the level of desired cooling. Temperature sensors may be placed at preferred locations in prosthetic socket or in the heat extraction subsystem to monitor temperature for safety as well as efficiency of the cooling system. Both of these inputs are provided as feedback to the microprocessor, and may be used in determining an optimal control signal for the fan to accomplish the desired temperature. This control signal is then sent to the fan driver to convert the control signal into the electrical power needed to drive the fan.

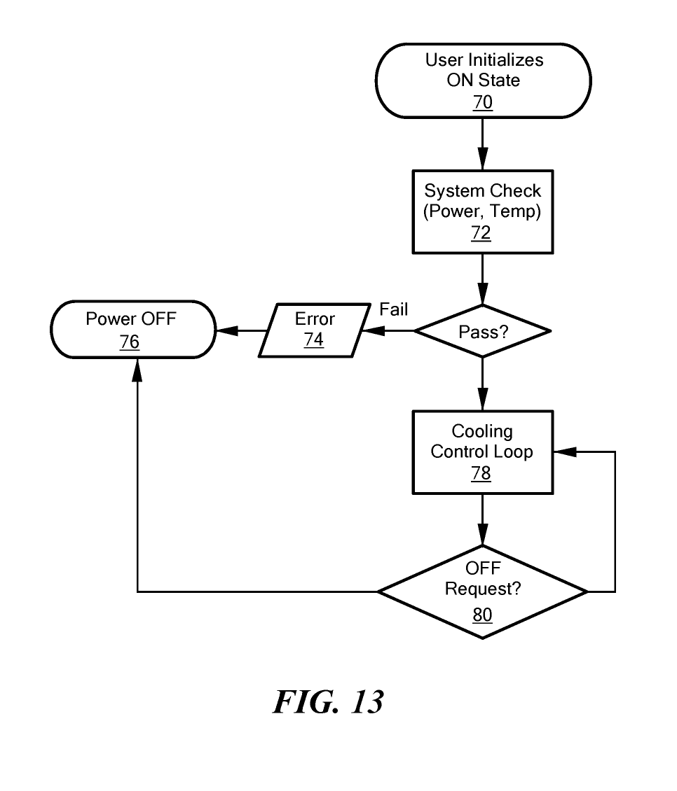

In one exemplary operational method of the for a prosthetic socket cooling system is now discussed with reference to FIGS. 13-15. The user turns the heat extraction subsystem device on using an ON/OFF button of the user interface step 70, FIG. 13. Once the ‘ON’ state is achieved, the device undergoes a system check, step 72. The system check establishes that all sensors, temperature readings, and input power are all within a pre-determined correct operating range. If any of these sub-systems fail the check, the thermal management device will go into an error state, step 74 and power down as shown, step 76. If all the subsystems pass, step 72, the device check, the thermal management device then enters into the cooling control loop, step 78. At this point, the cooling control loop internally processes and executes all requests for temperature adjustments. The device will remain within the cooling control loop until an ‘OFF’ request is received, step 80. See, e.g., the Main Control Loop Pseudocode in the Exemplary Code below.

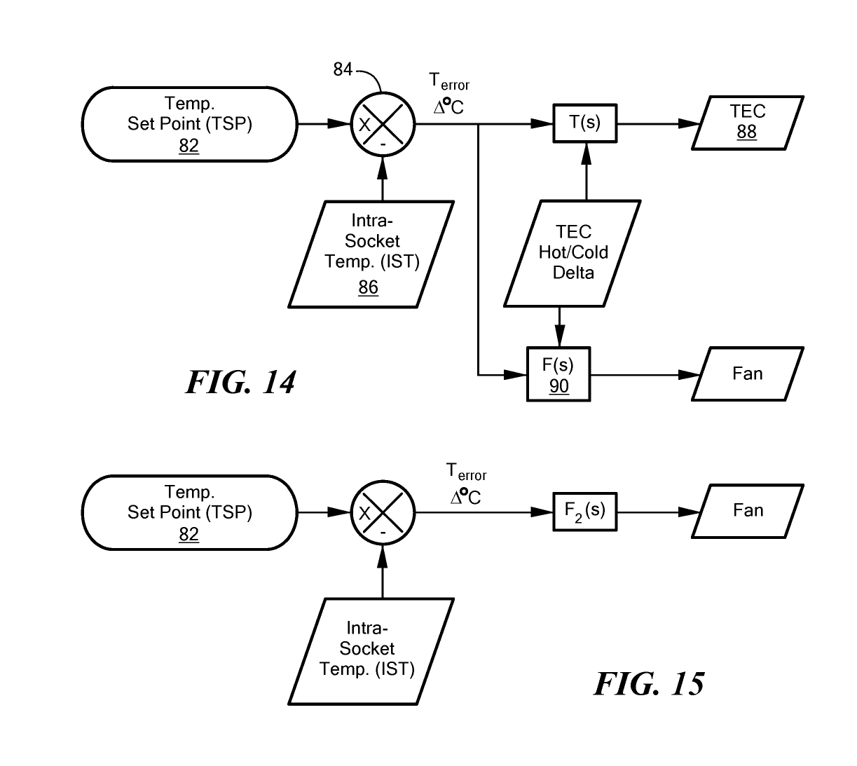

The temperature set point (TSP) 82, FIG. 14, is preferably established based on input from the user, e.g., using lower temperature or increase temperature button. The TSP is then compared, step 84 to the Intra-Socket temperature (IST) step 86 to determine the difference, also known as the temperature error (Tₑᵣᵣₒᵣ), between the actual temperature (IST) and the desired temperature (TSP). The temperature error is then passed to the TEC transfer function, T(s), step 88. This transfer function takes in the temperature error, the ambient temperature outside the socket, as well as the temperature difference between the hot and cold side of the TEC. This function then computes the control signal that gives the TEC sufficient power to achieve the desired temperature while minimizing battery discharge rate. The control signal input Tₑᵣᵣₒᵣ may then be passed to another transfer function, F(s) 90, that computes a control signal that drives the fan at a certain RPM such that the TEC is able to perform at optimal performance for a given TSP, ambient temperature, and TEC hot- and cold-side temperatures. See, e.g., The Cooling Control Loop Pseudocode (TEC and FAN) in the Exemplary Code below.

If the TEC is not used, the temperature set point (TSP) FIG. 15 is established based on input from the user, e.g., using lower temperature or increase temperature button. The TSP is then compared to the Intra-Socket temperature (IST) to determine the difference, also known as the temperature error (Tₑᵣᵣₒᵣ), between the actual temperature (IST) and the desired temperature (TSP). The temperature error is then passed to the fan transfer function1, F₂(s). This transfer function takes in the temperature error (and other variables) and computes a control signal that drives the fan at a certain RPM such that the desired temperature is achieved. See, e.g., The Cooling Control Loop Pseudocode (Fan only) in the Exemplary Code below.

The following Exemplary Code is provided which can be executed by Controller and/or the MCU to carry out the calculations, steps and/or functions discussed above. Other equivalent algorithms and code can be designed by a software engineer and/or programmer skilled in the art using the information provided therein:

Exemplary Code:

Main Control Loop Pseudocode:

```
void function main
{
      Call system_Check, and retrieve the TRUE/FALSE result
      Set the value of system_normal equal to the TRUE/FALSE result
      if the value of system_normal is equal to TRUE
            Call Cooling_Control_Loop
      if the value of system_normal is equal to FALSE
            Call system_Error
      if the power button is pressed
            Call system OFF
}
boolean function system_Check
{
      read all temperature sensors
      read battery level
      if the value of the temperature_sensors is within the correct range
            AND  the battery level is within the correct range
            return TRUE
      else
            return FALSE
}
void function system_Error
{
      Enable error indicator
      Wait for several seconds
  Call system_OFF
}
void function system_OFF
{
      Turn Cooling System OFF
      Turn Controller OFF
}
```

Cooling Control Loop Pseudocode (TEC and Fan):

```
void function Cooling_Control_Loop
{
      Call get_Temperature_Error, retrieve the decimal number result
      Set the value of temperature_Error equal to the decimal number result
      Call get_TEC_Temperature_Difference, retrieve the decimal number result
      Set the value of TEC_Temperature_Difference equal to the decimal number result
      Call compute_FAN_OUTPUT, provide the temperature_Error and TEC_Temperature_Difference, and
retrieve the result
      Set the value of FAN_Control equal to the decimal number result
      Send the value of FAN_Control to the FAN driver
      .
      Call compute_TEC_OUTPUT, provide the temperature_Error and TEC_Temperature_Difference, and
retrieve the result
      Set the value of TEC_Control equal to the decimal number result
      Send the value of TEC_Control to the TEC driver
}
decimal function get_Temperature_Error
{
      read the temperature set point
      read the Intra-socket temperature
      temperature_Error is set equal to (temperature set point) minus (Intra-socket temperature)
      return temperature_Error
}
decimal function get_TEC_Temperature_Difference
{
      read the temperatures of the HOT and COLD side of the TEC
      TEC_Temperature_Difference is set equal to (HOT side temperature) minus (COLD side temperature)
      return TEC_Temperature_Difference
}
decimal function compute_TEC_OUTPUT(temperature_Error, TEC_Temperature_Difference)
{
TEC_Output is set equal to the result of a transfer function T(s), that computes a value given temperature_Error and
TEC_Temperature_Difference
return TEC_Output
}
decimal function compute_FAN_OUTPUT(temperature_Error, TEC_Temperature_Difference);
{
      FAN Output is set equal to the result of a transfer function F(s), that computes a value given
temperature_Error and TEC_Temperature_Difference
return FAN_Output
}
```

Cooling Control Loop Pseudocode (Fan Only):

```
void function Cooling_Control_Loop
{
      Call get_Temperature_Error, retrieve the decimal number result
      Set the value of temperature_Error equal to the decimal number result
      Call compute_FAN_OUTPUT, provide the temperature_Error, and retrieve result
      Set the value of FAN_Control equal to the decimal number result
      Send the value of FAN_Control to the FAN driver
}
decimal function get_Temperature_Error
{
      read the temperature set point
      read the Intra-socket temperature
      temperature_Error is set equal to (temperature set point) minus (Tntra-socket temperature)
      return temperature_Error
}
decimal function compute_FAN_OUTPUT(temperature_Error);
{
      FAN Output is set equal to the result of a transfer function F(s), that computes a value given
temperature_Error
return FAN_Output
}
```

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

Other embodiments will occur to those skilled in the art and are within the following claims.

What is claimed is:

1. A prosthetic socket cooling system comprising:
a strap for coupling about a limb fitted with a prosthetic socket;
one or more lengthy thermally conductive heat spreaders extending from the strap and at least partially seated underneath the prosthetic socket; and
a heat extraction subsystem coupled to at least one of the lengthy heat spreaders.

2. The system of claim 1 in which the heat spreaders are thin sheets with rounded corners.

3. The system of claim 1 in which the heat spreaders are made of copper, aluminum, or graphite, or other conductive material.

4. The system of claim 1 in which the heat spreaders include one or more heat pipes.

5. The system of claim 1 in which the strap includes a thermally conductive material.

6. The system of claim 1 in which the heat extraction subsystem includes a finned heat sink and a fan positioned to urge air through the fins of the heat sink.

7. The system of claim 6 in which the heat extraction subsystem further includes a TEC.

8. The system of claim 7 in which the TEC is coupled between the heat spreader and the heat sink.

9. The system of claim 7 in which the heat extraction subsystem further includes a user interface, an electronic section, and a battery for powering the TEC, the fan, and the electronics section.

10. The system of claim 9 further including a housing about the fan, the TEC, the heat sink, the user interface, the electronics section, and the battery.

11. The system of claim 9 in which the electronics section further includes a controller subsystem and one or more temperature sensors.

12. The system of claim 11 in which the controller is configured to operate the fan and/or the TEC based on signals from the user interface and the temperature sensors.

13. A method of cooling a prosthetic socket, the method comprising:
strapping a plurality of spaced lengthy thermally conductive heat spreaders about a limb fitted with a prosthetic socket;
placing the lengthy thermally conductive heat spreaders underneath the prosthetic socket;
coupling a heat extraction subsystem to at least one of the lengthy heat spreaders; and
operating the heat extraction subsystem to drive heat from the prosthetic socket to the external environment via the heat spreaders and the heat extraction subsystem.

14. The method of claim 13 in which the heat spreaders are thin sheets with rounded corners.

15. The method of claim 13 in which the heat spreaders are made of copper, aluminum, or graphite or other thermally conductive materials.

16. The method of claim 13 in which the heat spreaders include one or more heat pipes.

17. The method of claim 13 in which the strap includes thermally conductive material.

18. The method of claim 13 in which the heat extraction subsystem includes a finned heat sink and a fan positioned to urge air through the fins of the heat sink.

19. The method of claim 18 in which the heat extraction subsystem further includes a TEC.

20. The method of claim 19 in which the TEC is coupled between the heat spreaders and the heat sink.

21. The method of claim 20 in which the heat extraction subsystem further includes a user interface, an electronic section, and a battery for powering the TEC, the fan, and the electronics section.

22. The method of claim 21 further including a housing about the fan, the TEC, the heat sink, the user interface, the electronics section, and the battery.

23. The method of claim 21 in which the electronics section further includes a controller subsystem and one or more temperature sensors.

24. The method of claim 23 in which the controller is configured to operate the fan and/or the TEC based on signals from the user interface and the temperature sensors.

25. A system for cooling a prosthetic socket on a limb, the system comprising:
a strap for coupling about the limb above the prosthetic socket;
one or more thermally conductive heat spreaders associated with the strap; and
at least one heat extraction subsystem carried by the strap.

26. The system of claim 25 in which the heat spreaders extend from the strap and can be disposed underneath the prosthetic socket.

27. The system of claim 25 in which the heat spreaders are with a part of the strap and/or form sections of the strap.

28. The system of claim 25 in which the heat spreaders are thin sheets with rounded corners.

29. The system of claim 25 in which the heat spreaders are made of copper, aluminum, or graphite or other conductive material.

30. The system of claim 25 in which the heat extraction subsystem includes a finned heat sink and a fan positioned to urge air through the fins of the heat sink.

31. The system of claim 30 in which the heat extraction subsystem further includes a TEC.

32. The system of claim 31 in which the TEC is coupled between the heat spreader and the heat sink.
